(19) United States
(12) Patent Application Publication
(10) Pub. No.: US 2017/0128695 A1
(43) Pub. Date: May 11, 2017

(54) THREE LUMEN BALLOON CATHETER APPARATUS

(71) Applicant: Paul Speiser, Wien (AT)

(72) Inventor: Paul Speiser, Wien (AT)

(21) Appl. No.: 15/382,379

(22) Filed: Dec. 16, 2016

Related U.S. Application Data
(63) Continuation-in-part of application No. 14/647,073, filed on May 22, 2015.

(30) Foreign Application Priority Data
May 29, 2013 (EP) 13169644.5

(51) Int. Cl. A61M 25/00; A61B 6/00; A61B 5/00; A61M 25/10

(52) U.S. Cl. CPC A61M 25/003; A61M 25/10181; A61B 6/481; A61B 5/4325; A61B 2010/0074

(57) ABSTRACT

The present invention relates to a catheter, in particular to a balloon bearing, three lumen catheter for dispensing diagnostic fluids into a body cavity and retrieving a sample of the diagnostic fluid.

### CROSS-REFERENCE TO RELATED APPLICATIONS

This application claims the benefit of priority under 35 U.S.C. §120 from, and is a continuation-in-part of, U.S. patent application Ser. No. 14/647,073, filed May 22, 2015, which is the U.S. national stage of International Patent Application No. PCT/EP2014/061246, filed May 30, 2014, and under 35 U.S.C. §119 from European Patent Application No. 13169644.5, filed May 29, 2013. The disclosures of the foregoing applications are incorporated herein by reference in their entirety.

### FIELD OF THE INVENTION

The present invention relates to a catheter, in particular to a balloon-bearing three lumen catheter for simultaneous dispensing diagnostic fluids into a body cavity and retrieving a sample of the diagnostic fluid at the same time and a catheter apparatus employing same.

### BACKGROUND OF THE INVENTION

Ovarian cancer is the leading cause of death from gynecologic malignancy in western civilized countries, with an estimated prevalence in Europe and the U.S.A. of 752,600 in 2007 and 59,828 deaths yearly. Treatment and survival of patients depend primarily on the stage of the disease. Of all ovarian cancer patients, only 25% are diagnosed at an early stage while the tumor is confined to the pelvis. In these cases the five-year survival rate is 80% to 90% and the disease can often be cured by a combination of surgery and chemotherapy, and this stage is increasingly frequently referred to as type I ovarian cancer. Unfortunately, almost 75% of women affected have advanced stage disease with metastatic spread throughout the abdominal cavity or to retroperitoneal lymph nodes at the time of diagnosis. Five-year survival rates fall to 19%-32% for advanced disease, despite maximum surgical effort and combination chemotherapy. This so called type II ovarian cancer is the deadliest cancer in women, even more aggressive than lung cancer.

Diagnostic procedures which require a non-surgical entry into the uterus are well known. One such procedure known as hysterosalpingography is a radiographic method for imaging the anatomical structures of the uterus and fallopian tubes. Hysterosalpingography involves inserting a fine flexible catheter through the cervical canal and injecting a contrast medium, such as an iodinated fluid, into the uterus. Radiography is then carried out to provide imaging information pertaining to the subject uterus.

Another well-known diagnostic procedure which entails a non-surgical entry into the uterus is called saline contrast hysterosonography. This procedure also employs a fine flexible catheter that is inserted into the cervical canal of the uterus. The catheter in this procedure enables the physician or technician to inject a sterile saline solution into the uterus to expand it so that an ultrasound scanner can be used to sonographically observe the uterus.

The uterine cavity is in a sense a virtual space, because the anterior and posterior walls of the uterus are in direct contact with each other. Particularly in postmenopausal but also in non-pregnant premenopausal women, the body of the uterus containing the uterine cavity is very small. The cavity itself has a triangular shape measuring only approx. 3 cm in length and approx. 2 cm at the roof. When a fluid gets dispensed into the cavity, it immediately evacuates through the fallopian tubes into the peritoneal cavity and the dispensed fluid cannot be retrieved anymore. Furthermore, on aspiration the openings of catheters tend to get obstructed by loose tissue from the lining of the cavity, called endometrium, or by blood clots frequently present in the uterine cavity.

Cells shed from the lining of the fallopian tube and ovary get transported into the cavity and are of great interest for detecting premalignant and malignant changes in these tissues. These cells are extremely few in number, making it difficult to sample them. All sampling devices for the uterine cavity to date are designed to sample tissue of the lining of the uterine cavity to detect premalignant or malignant changes of the endometrium. For the detection of the minute number of cells shed from the lining of the fallopian tubes and ovaries which have found their way into the uterine cavity, a sampling device is desired that would allow retrieving those loose cells with as little contamination as possible of endometrial tissue.

WO03033045 discloses a catheter with suction capability to obtain a biosample. Specifically, the catheter is designed for collecting prostatic fluid from the prostatic urethra and/or the membranous urethra. To obtain a biosample, the catheter exhibits biosample entry ports (25) which are positioned along the elongated body. Additionally the catheter has a urinary drainage port (26). Therefore, the catheter according to WO03033045 is not suitable to simultaneously dispense a diagnostic fluid and retrieve a sample of the dispensed fluid at the same time.

PCT/EP2013/051899 discloses a non-invasive method for the diagnosis of adenocarcinoma or their precursor lesions of ovaries, fallopian tubes and endometrial lining in a female subject by analyzing cells of said subject using a uterine catheter designed for non-invasive rinsing of the uterine cavity and collecting a sample of the rinse. One problem associated with the catheters of this design is that they have a closed tip which makes it difficult to retrieve a sample of the dispensed fluid, representing a lavage of the fallopian tubes and uterus. Therefore, a catheter which allows for simple and reproducible retrieving a sample from the dispensed fluid is desirable.

### SUMMARY OF THE INVENTION

To overcome the problems of prior catheters, the present catheter comprises a very short tip to fit into the small space of the uterine cavity and allows for simultaneous flushing of fluid into the uterine cavity and proximal tubes and aspirating the fluid at the same time, preventing the fluid from evacuating through the fallopian tubes. To reduce the risk of obstruction, the channels have more than one opening, preferably pointing in different directions. To prevent the fluid from back flowing through the cervical canal, a small balloon is preferably included.

It is an aspect of the invention to provide an improved catheter which can be used for non-surgical entry into a uterus to dispense a diagnostic fluid therein and to retrieve a sample of the dispensed fluid simultaneously representing a lavage of the fallopian tubes and uterus. The catheter comprises an elongated body having two single lumens extending continuously from a proximal end up to the top of the tip at the distal end and a third lumen extending continuously from the proximal end to an inflatable balloon.

The present catheter preferably includes an elongated body having a proximal end and a distal end with a distal face. The elongated body further comprises first and second lumens extending from the proximal end of the elongated body to the distal end of the elongated body. The first lumen has a first opening at the distal face of the elongated body for dispensing a diagnostic fluid into the interior of the subject's uterus, while the second lumen has a second opening at the distal face of the elongated body for retrieving a sample of the dispensed fluid from the interior of the subject's uterus. The first opening and second opening preferably each have a diameter of 800 μm to 1500 μm.

The catheter further preferably includes a balloon disposed proximally with respect to the distal end of the elongated body for sealing the cervical canal in a fluid-tight manner when inflated, with a third lumen extending from the proximal end of the elongated body and having an opening in communication with the balloon in order to inflate the balloon. Preferably, the center of the balloon is at a distance of 3 mm to 15 mm from the distal end of the elongated body, and the maximum volume of the balloon is 500 μl to 1500 μl. The third lumen can advantageously extend separately from the first and second lumens from a point proximal to the connecting element to the proximal end of the elongated body, where the proximal end of the third lumen has a closure device.

In a preferred embodiment, the first and second lumens each have at least one side opening between the distal end of the elongated body and the balloon in addition to the openings at the distal face. These side openings each preferably have a diameter of from 700 μm to 1400 μm.

At a proximal end of the elongated body, the present catheter further preferably comprises a connecting element in which the first lumen, the second lumen, and the third lumen are fixed. The first lumen and the second lumen preferably extend separately from each other as flexible tubes from a point proximal to the connecting element toward the proximal end of the elongated body. The proximal ends of the first lumen and the second lumen can each advantageously be connected to a syringe.

### BRIEF DESCRIPTION OF THE DRAWINGS

FIG. 1 is a top plan view of the present catheter.

FIG. 2a is a top plan view of the tip.

FIG. 2b is a bottom plan view of the tip.

FIG. 2c is a side elevation of the catheter, showing the distal face of the tip.

FIG. 2d is a section view along line A-A of FIGS. 2a and 2b.

FIG. 2e is a section view along line B-B of FIG. 2c.

FIG. 2f is a section view along line C-C of FIGS. 2a and 2b.

FIG. 3 is a view of the connecting element.

FIG. 4 is a top plan view of the proximal end of the catheter.

FIG. 5 is a side elevation view of an applicator assembly.

FIG. 6 is a side elevation view of two syringes fixed in the applicator assembly.

FIG. 7 is a side elevation view of the applicator assembly with an adapter.

FIG. 8 is a side elevation view of applicator assembly with the adapter and a lip.

FIG. 9a is a top plan view of the catheter.

FIG. 9b is an enlarged, top plan view of the distal end of the catheter.

FIG. 9c is a side elevation of the catheter, showing the distal face of the tip.

FIG. 9d is a section view of the distal end of the catheter along line A-A of FIG. 9b.

### DETAILED DESCRIPTION OF THE INVENTION

The present invention relates to a catheter useful for non-surgical entry into a uterus to dispense a diagnostic fluid therein and to retrieve simultaneously a sample from said dispensed fluid representing a lavage of the fallopian tubes and uterus, the catheter comprises:

an elongated body (1) having three lumens (4, 5 and 6),

a balloon (9) disposed marginally adjacent to the distal end (3) of the elongated body (1) for fluid sealing the interior of the subject's uterus; wherein

lumens (4 and 5) extend from the proximal end (2) up to the distal end (3) of the elongated body, and

lumen (6) extends from the proximal end (2) to the distal end (3) of the elongated body with an opening that preferably lies proximally from the distal end (3) for inflating the balloon (9), and

lumen (4) has an opening (4′) at the top of the tip (7) for dispensing a diagnostic fluid into the interior of a subject's uterus and tubes, and

lumen (5) has an opening (5′) at the top of the tip (7) for retrieving a sample of said dispensed fluid of the interior of a subject's uterus representing a lavage of the fallopian tubes and uterus simultaneously.

An embodiment of the invention is the catheter as described above, wherein the lumens (4 and 5) have additionally side openings (8) between the top of the tip (7) and the balloon (9). One of the side openings (4″) communicates with lumen (4), while the other side opening (5″) communicates with the lumen (5).

In some embodiments, the elongated body extending from the proximal end (2) to the distal end (3) has a length of any one of 250 mm to 350 mm, 260 mm to 340 mm, 270 mm to 330 mm, 280 mm to 320 mm, or 290 mm to 310 mm. In some embodiments, the elongated body extending from the proximal end (2) to the distal end (3) is at least any one of 250 mm, 260 mm, 270 mm, 280 mm, 290 mm, 300 mm, 310 mm, 320 mm, 330 mm, 340 mm or 350 mm. In some embodiments, the elongated body extending from the proximal end (2) to the distal end (3) has a length of 300 mm to 320 mm (e.g., 310 mm). In some embodiments, the elongated body extending from the proximal end (2) to the distal end (3) has a length of any one of 290 mm, 300 mm, 310 mm, 320 mm, or 330 mm.

The elongated body may be circular or of squared shape with rounded or smoothed edges. The diameter of the elongated body may be any one of 2500 μm; 2600 μm; 2700 μm; 2800 μm; 2900 μm; 3000 μm; 3100 μm; 3200 μm; 3300 μm, 3400 μm, 3500 μm 3600 μm, 3700 μm, 3800 μm 3900 μm, or 4000 μm (e.g. 3300 μm). If the elongated body is of squared shape with rounded or smoothed edges the side length of the square may be any one of 2500 μm; 2600 μm; 2700 μm; 2800 μm; 2900 μm; 3000 μm; 3100 μm; 3200 μm; 3300 μm, 3400 μm, 3500 μm 3600 μm, 3700 μm, 3800 μm 3900 μm, or 4000 μm (e.g. 3300 μm).

The balloon (9) of the catheter described herein is adjacent to the distal end (3) of the elongated body. For example, the balloon (e.g. the distal outer circumference of the balloon when inflated) may be at a distance of any one of up to 3 mm, 4 mm, 5 mm, 6 mm, 7 mm, 8 mm, 9 mm, 10 mm, 11 mm, or 12 mm from the distal end. In some embodiments, the balloon (e.g. the distal outer circumference of the balloon when inflated) is at a distance from the distal end (3), which is 3 mm to 12 mm, 3 mm to 9 mm, 3 mm to 8 mm, 3 mm to 7 mm, 3 mm to 6 mm, 4 mm to 12 mm, 4 mm to 10 mm, 4 mm to 8 mm, 5 mm to 12 mm, 5 mm to 10 mm, 5 mm to 8 mm. In some embodiments the distance of the distal end (3) to the center of the balloon is any one of 3 mm to 15 mm, 3 mm to 12 mm, 3 mm to 9 mm, 4 mm to 15 mm, 4 mm to 12 mm, 4 mm to 9 mm, 5 mm to 15 mm, 5 mm to 12 mm, 5 mm to 9 mm, 6 mm to 15 mm, 6 mm to 12 mm, or 6 mm to 9 mm.

Exemplary sizes of the balloon are balloons with a diameter of 5 mm to 15 mm, 5 mm to 12 mm, 5 mm to 10 mm, 5 mm to 8 mm, 7 mm to 15 mm, 7 mm to 13 mm, 7 mm to 11 mm, 9 mm to 15 mm, 9 mm to 13 mm, 9 mm to 11 mm, 7 mm to 9 mm or 6 mm to 10 mm. In some embodiments, the diameter of the balloon is any one of 7, 8, 9, 10, 11, 12 or 13 mm. The maximum volume of the balloon in its inflated state may be any one of 500 μl, 600 μl, 7000, 800 μl, 900 μl, 10000, 1100 μl, 1200 μl, 1300 μl, 1400 μl or 1500 μl. In some embodiments, the maximum volume of the balloon is any one of 500 μl to 1500 μl, 600 μl to 1400 μl, 700 μl to 1300 μl, 800 μl to 1200 μl, or 900 μl to 1100 μl (e.g. 1000 μl).

The elongated body of the catheter described herein may comprise a taped area around the balloon (e.g. in order to hold the balloon in a fixed position), or the balloon may otherwise be secured to the catheter in ways known to the art. The taped area may extend from the balloon (e.g. the outer circumference of the balloon when inflated) along the elongated body of the catheter for any one of 1-6 mm, 2-6 mm, 3-6 mm, 4-6 mm, 5-6 mm, 1-5 mm, 2-5 mm, 3-5 mm, 1-4 mm, 1-3 mm or 2-4 mm. In some embodiments, the taped area covers the elongated body of the catheter for any one of 1, 2, 3, 4, 5, 6 mm (e.g. 3 mm) on both sides of the balloon when inflated (e.g. on the distal and proximal side of the balloon along the elongated body).

The catheter described herein comprises two lumens (4) and (5), each having an opening (4′ and 5′) on the distal face (30) at the top of the tip (7), and in some embodiments one or more side openings (4″ and 5″) between the distal face (30) and the balloon (9) (e.g. the distal outer circumference of the balloon when inflated). In some embodiments lumen (4) has at least one side opening (4″) in addition to the opening (4′) at the top of the tip, and lumen (5) has at least one side opening (5″) in addition to the opening (5′) at the top of the tip. In some embodiments, lumen (4) has one side opening (4″) and lumen (5) has one side opening (5″), while in other embodiments, lumen (4) has at least two side openings and lumen (5) has at least two side openings. The diameter of lumen (4) and 5 may be any one of 800 μm, 900 μm, 1000 μm, 1100 μm, 1200 μm, 1300 μm, 1400, μm, or 1500 μm (e.g. 1200 μm). Accordingly, the diameter of the openings (4′ and 5′) at the top of the tip may be any one of 800 μm, 900 μm, 1000 μm, 1100 μm, 1200 μm, 1300 μm, 1400 μm, or 1500 μm (e.g. 1200 μm). In some embodiments the diameter of lumen (4) and lumen (5) are the same. The diameter of the side openings (4″/5″) of lumen (4) and 5 may be any one of 700 μm 800 μm, 900 μm, 1000 μm, 1100 μm, 1200 μm, 1300 μm, or 1400 μm (e.g. 1100 μm). The distance of the distal end of the catheter and the side opening (e.g. the center of the side opening) may be any one of 750 μm to 10 mm, 750 μm to 5 mm, 750 μm to 3 mm, 750 μm to 1500 μm, 1 mm to 10 mm, 1 mm to 7 mm, or 1 mm to 5 mm.

In some embodiments the elongated body of the catheter described herein does not have any side openings (e.g. openings in the side wall of the elongated body) between the balloon (9) (e.g. the proximal outer circumference of the balloon when inflated) and the proximal end (2).

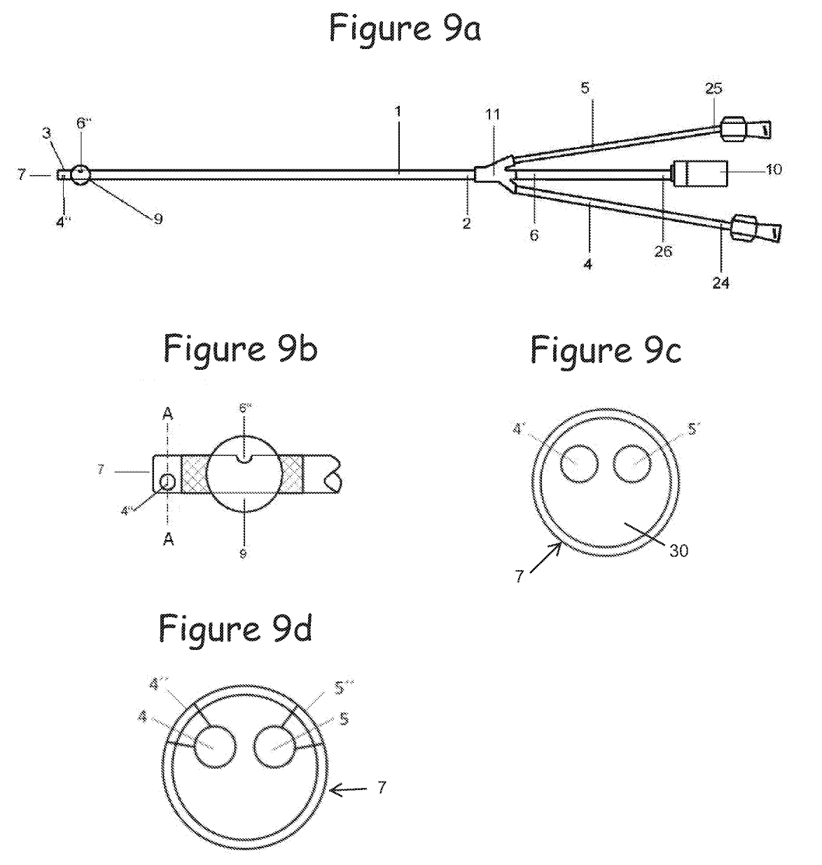

A further embodiment of the invention, shown in FIG. 9, is the catheter as described above, further including a mechanism for connecting syringes to the catheter. The catheter described herein may further comprise a connecting element (11) at the proximal end (2) of the elongated body in which lumens 4, 5 and 6 are fixed. In some embodiments the three lumens extend separately from each other proximal of the connecting element (11) to their respective proximal ends (24, 25, 26). In some embodiments, lumens 4 and 5 extend from the connecting element to their proximal end (24 and 25) as tubes (e.g. tubes with dimensions of 1.5×2.7 mm). In some embodiments the tubes extending from the connecting element (11) to their proximal end (24 and 25) are separate flexible tubes. In some embodiments, lumens 4 and 5 may be connected to syringes at their proximal end (24, 25). In some embodiments, the length of lumen (4) and 5, and accordingly the respective tubes, from the connecting element (11) to the proximal end of lumens 4 (proximal end 24) and 5 (proximal end 25) is any one of between 150 mm to 250 mm, 150 mm to 230 mm, 150 mm to 200 mm, 170 mm to 250 mm, 170 mm to 230 mm, or 170 mm to 200 mm. In some embodiments, the length is any one of 180 mm, 190 mm, 200 mm, 210 mm or 220 mm. Lumen 6 may also extend proximally from the connecting element (11) to its proximal end (26) as flexible tube. In some embodiments, the length from the tip of the elongated body (7) to the proximal end (26) of lumen 6 is any one of 420 mm to 540 mm, 440 mm to 520 mm, 460 mm to 500 mm, 470 mm to 490 mm (e.g. 480 mm).

A further embodiment of the invention is the catheter as described above, wherein the third lumen has a closure device (10) at the proximal end of lumen 6 (26) for closing the lumen upon filling the balloon with said closure device at the proximal end. A further embodiment of the invention is the catheter as described above, wherein said closure device (10) is a blocking valve.

The third lumen (6) extends from its proximal end (26) to an opening leading to the balloon (6″). In some embodiments, lumen 6 ends in the opening leading to the balloon (6″). In some embodiments, lumen 6 extends further to the distal end (3) of the elongated body (e.g. extending any one of 1, 2, 3, 4, 5, 6, mm distally from the side opening 6″). In some embodiments, lumen 6 extends all the way to the distal end (3) of the elongated body. In some embodiments, lumen 6 has an opening (6′) at the top of the tip (7). In some embodiments, the opening (6′) of lumen 6 at the top of the tip (7) is closed/sealed (e.g. covered with tape) to allow inflation of the balloon:

A further embodiment of the invention is a kit for dispensing a diagnostic fluid and to retrieve a sample from the dispensed fluid from a subject's uterus, comprising
a catheter as described above,
an application assembly for connecting two syringes; and
an adapter for simultaneously moving the two plungers in opposite directions.

A further embodiment of the invention is the kit as described above, wherein said adapter has a lip (19).

A further embodiment of the invention is the use of a kit as described above for screening a female population being at risk of endometrial cancer or suffering from premalignant changes of the endometrium or endometrial cancer.

A further embodiment of the invention is the use of a kit as described above for screening a female population being at risk of ovarian cancer or suffering from premalignant changes of the ovary or ovarian cancer.

A further embodiment of the invention is the use of a kit as described above for screening a female population being at risk of cancer of the fallopian tube or suffering from premalignant changes of the tube or tubal cancer.

A further embodiment of the invention is a non-invasive method of collecting a sample for ex vivo diagnostic purposes by rinsing the uterine cavity and optionally the fallopian tubes of a subject with a lavage fluid and retrieving a cell sample from the rinse. The lavage fluid is dispensed through one of the lumens (4 or 5), i.e. through the openings 4′ and/or 4″ or 5′ and/or 5″, and is collected through other lumen and associated opening(s). Preferably, the fluid is dispensed through one lumen opening(s) and simultaneously collected (e.g., by suction) through the other lumen opening(s).

The invention is now described by reference to various specific embodiments which are shown in the attached drawings. It is to be clearly understood that these embodiments are shown for purpose of illustration only and are not limiting.

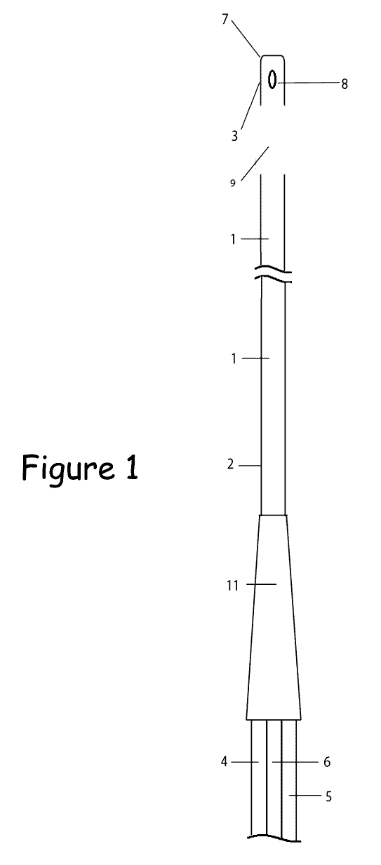

Attention is first directed to FIG. 1, which shows a catheter with an elongated body (1) and three internal lumens (4, 5 and 6). Two lumens (4 and 5) extend to the distal end (3) of the elongated body (1) with openings (4′ and 5′) at the top of the tip at the distal end. “Distal end” refers to the end furthest from the person holding the apparatus, and “proximal end” refers to the end closest to the holder of the apparatus. Optionally the lumens (4 and 5) have side openings (8) adjacent to the tip of the catheter. The additional side openings (8) are located close to the tip of the catheter, in particular between the tip of the catheter and the balloon.

The third lumen (6) ends relatively close to the distal end of the elongated body (1) where the balloon is disposed and communicates through the bore with the interior of the balloon (9).

The balloon is adjacent to the tip of the catheter and seals the cervix which is a prerequisite to obtain a sample of the lavage solution. The balloon is made of expandable material. When deflated, the balloon (9) lies closely adjacent to the body, so as not to interfere with the insertion or withdrawal of the catheter. When the balloon is inflated by providing e.g. normal saline through the lumen (6) it seals the cervical canal.

It is further required, that the elongated body does not contain any additional openings between the balloon and the proximal end of the elongated body (1). Additional openings between the balloon and the proximal end of the elongated body would be conflicting with the aim of the invention to lavage the uterus and to obtain a sample of the lavage fluid at the same time.

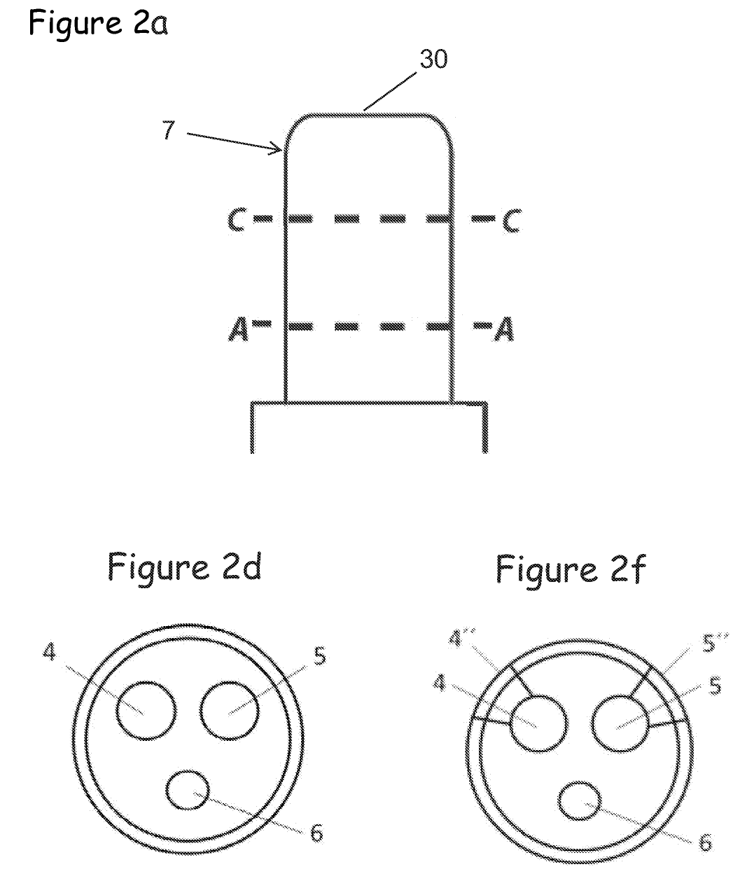

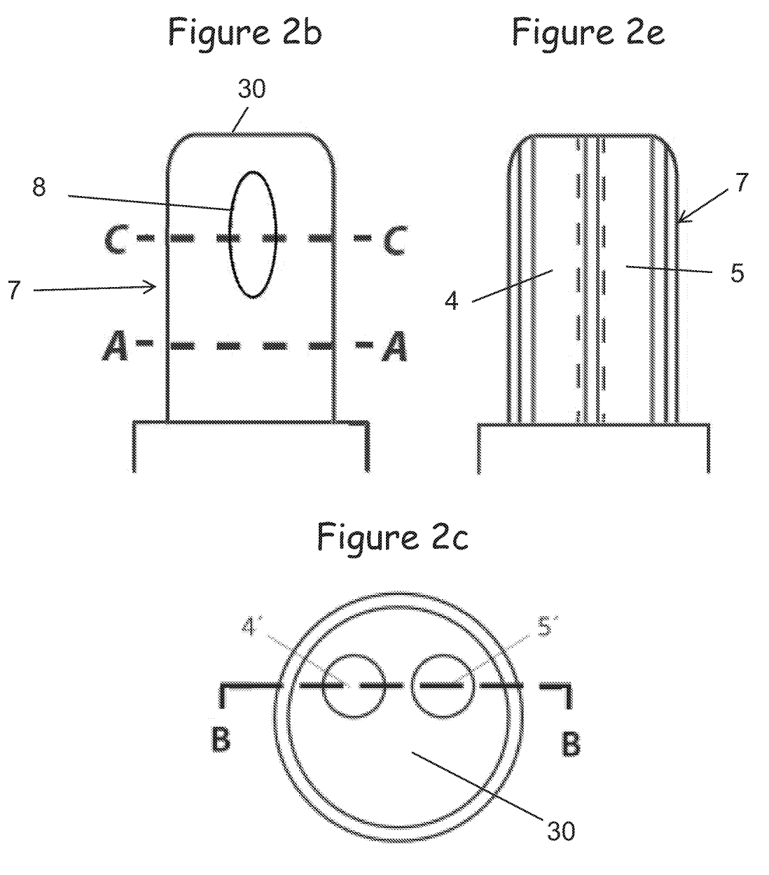

FIG. 2 shows frontal view, lateral view, top view and sections (A-A, B-B, C-C) of the tip. Section B-B exhibits the two openings (4′ and 5′) of lumens (4 and 5) on the top of the tip (7). Section A-A exhibits the three lumens within in the elongated body (1).

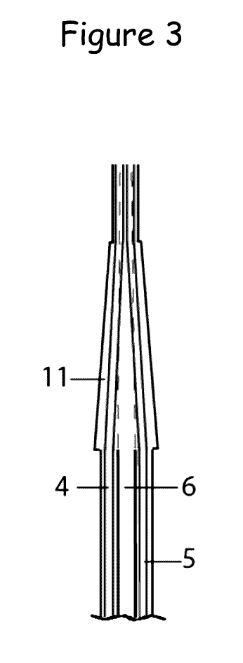

FIG. 3 shows a connecting element (11) at the proximal end (2) of the elongated body (1) in which the lumens (4, 5 and 6) are fixed.

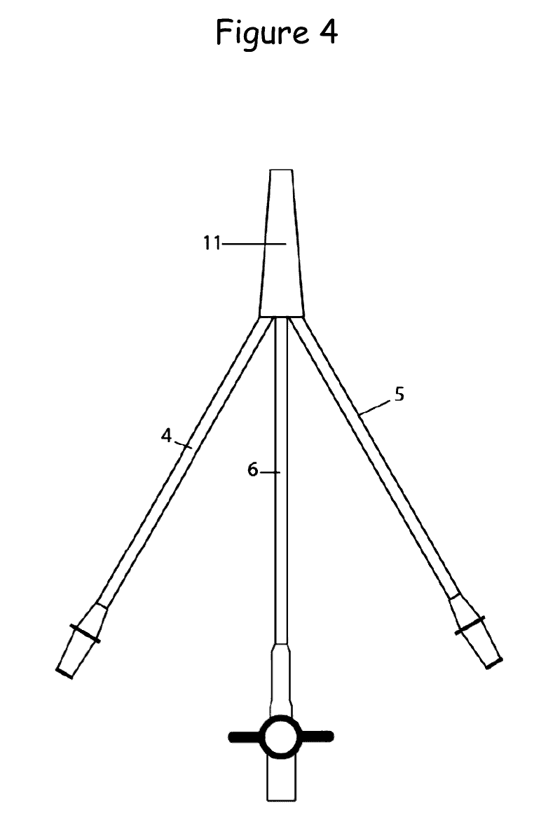

FIG. 4 shows the proximal end of the catheter, wherein the three lumens are extending the connecting element (11). The lumens (4 and 5) may be connected to syringes. The lumen (6) may bear a closing element (10), such as for example a blocking valve or a plug. The connection is normally made by supplying small coupling elements to connect the lumens to the syringes.

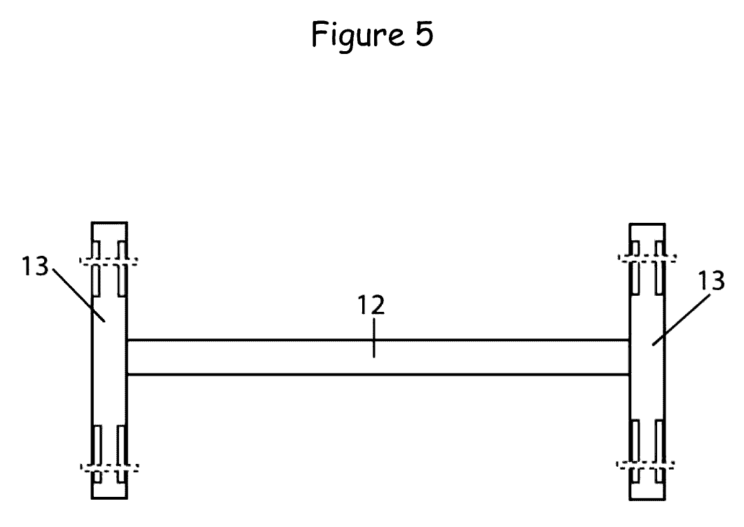

FIG. 5 shows an applicator assembly for connecting two syringes. The applicator assembly consists of at least one guide bar (12) and two ports (13). In the ports (13) the flanges 16 and 17 of the syringes are affixed.

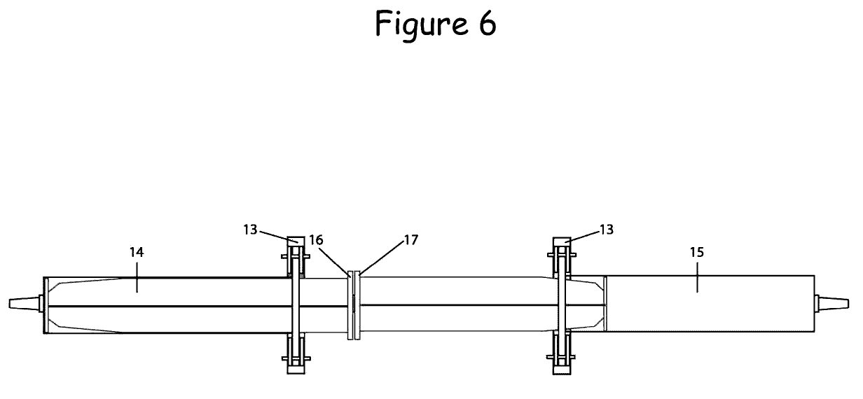

FIG. 6 shows two syringes (14) and (15) affixed in the applicator assembly. One syringe (14) is empty, thus the plunger is in the zero position. The other syringe (15) is filled with a lavage fluid, for example with normal saline.

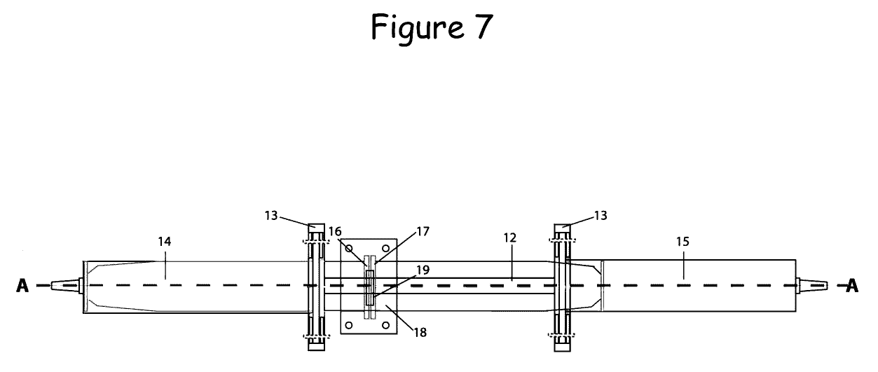

FIG. 7 shows the applicator assembly with an adapter 18 for connecting the flanges 16 and 17 of the syringes (14) and (15).

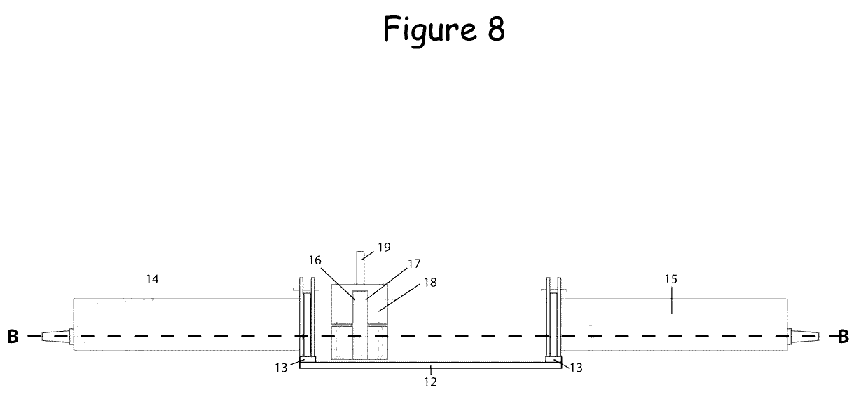

FIG. 8 shows the applicator assembly with the adapter 18, wherein the adapter has a lip (19) which allows for the application of the lavage fluid. Upon dispensing the lavage fluid, the plunger of syringe (15) is moved to the zero position and the plunger of syringe (14) is pulled back. By pushing on the plunger of the lavage fluid containing syringe, the fluid is slowly syringed into the uterine cavity and tubes. Simultaneously the plunger of the empty syringe is gently pulled out, sucking the fluid from the uterine cavity and tubes. While one tube slowly empties, the other slowly fills up. Thus, the adapter 18 with the lip (19) allows for simultaneous rinsing and sucking.

FIG. 9a) to d) show a catheter with an elongated body (1) of a length of 310 mm and a diameter of 3300 μm. The catheter has three internal lumens (4, 5 and 6). Two lumens (4 and 5) extend to the distal end (3) of the elongated body (1) with openings (4′ and 5′) at the top of the tip (7) at the distal end with a diameter of 1200 μm. Optionally, two lumens (4 and 5) have side openings (4″ and/or 5″) with a diameter of 1100 μm and the distance from the distal end of the elongated body (3) and the center of the side opening is 1 mm to 3 mm.

In one embodiment the third lumen (6) ends at the distal end of the elongated body (1) and has a side opening or bore (6″) where the balloon is disposed and communicates through the bore with the interior of the balloon (9). The opening 6′ at the top of the tip has a diameter of 800 μm and is closed with tape

The balloon has a diameter between 8 to 10 mm and its distal outer circumference when inflated is 6 mm away (its center is 8 to 10 mm away) from the tip of the catheter (7) and seals the cervix which is a prerequisite to obtain a sample of the lavage solution. The balloon is made of expandable material. When deflated, the balloon (9) lies closely adjacent to the body, so as not to interfere with the insertion or withdrawal of the catheter. When the balloon is inflated by providing e.g. normal saline through the lumen (6) it seals the cervical canal

It is further required, that the elongated body does not contain any additional openings (e.g. side openings) between the balloon (9) and the proximal end (2) of the elongated body (1).

According to a specific example, two 10 ml syringes—one of them containing 10 ml of normal saline—are connected to the two lumens (4 and 5). By pushing on the lip (19) the normal saline containing syringe (15), the fluid is slowly syringed into the uterine cavity and tubes through one of the lumens (4 or 5). Simultaneously, the plunger of the empty syringe (14) is gently pulled out, sucking the fluid from the uterine cavity and tubes through the other lumen. While one tube slowly empties, the other slowly fills up. After the lavage is finished, a cap is put on the filled syringe and the syringe is sent into the laboratory for diagnosis.

The catheters used in this procedure typically have means for sealing off the uterus before or during injection of the fluid to prevent backflow into the vagina. One such means includes an inflatable intrauterine balloon made from an elastomeric material which is disposed adjacent the distal tip of the catheter.

The balloon catheter is operated by inserting the distal tip thereof through the cervical canal and into the uterus with the intrauterine balloon deflated. The insertion of the distal tip operates to position the deflated intrauterine balloon in the uterus or cervical canal. Once positioned, the inflation syringe is used to inflate the balloon with air or saline to create a seal in the cervical canal and the injection syringe is used to inject the desired diagnostic fluid into the uterus.

The uterus and the fallopian tubes are rinsed with the diagnostic fluid. Due to the specific design of the catheter, a sample of said diagnostic fluid could be obtained at the same time which is useful for further analysis.

The development of an alternate and new test to detect ovarian cancer or tubal cancer or endometrial cancer and their precursor lesions is imperative, in particular serous ovarian cancer or tubal cancer or endometrial cancer or disease of early stages. Thus, the present invention is a valuable contribution to a convenient, safe and cost-effective test possibly used for early diagnosis or in a screening program looking for cancer or precancerous lesions in women who have no symptoms of the disease.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

1. A catheter useful for non-surgical entry into a uterus of a subject to dispense a diagnostic fluid therein and to the fallopian tubes and to retrieve a sample from the dispensed fluid, the catheter comprising:
an elongated body having a proximal end and a distal end with a distal face, the elongated body further comprising:
a first lumen extending from the proximal end of the elongated body to the distal end of the elongated body, wherein the first lumen has a first opening at the distal face of the elongated body for dispensing a diagnostic fluid into the interior of the subject's uterus;
a second lumen extending from the proximal end of the elongated body to the distal end of the elongated body, wherein the second lumen has a second opening at the distal face of the elongated body for retrieving a sample of the dispensed fluid from the interior of the subject's uterus; and
a third lumen having a proximal end and a distal end; and a balloon disposed proximally with respect to the distal end of the elongated body for sealing the cervical canal in a fluid-tight manner when inflated
wherein the third lumen extends from the proximal end of the elongated body and has an opening in communication with the balloon in order to inflate the balloon.

2. The catheter of claim 1, wherein the center of the balloon is at a distance of 3 mm to 15 mm from the distal end of the elongated body.

3. The catheter of claim 1, wherein the maximum volume of the balloon is 500 μl to 1500 μl.

4. The catheter of claim 1, wherein the first opening and the second opening of the first and the second lumens each have a diameter of 800 μm to 1500 μm.

5. The catheter of claim 1, wherein the first lumen has at least one side opening between the distal end of the elongated body and the balloon in addition to the first opening at the distal face and the second lumen has at least one side opening between the distal end of the elongated body and the balloon in addition to the second opening at the distal face.

6. The catheter of claim 5, wherein the side openings each have a diameter of 700 μm to 1400 μm.

7. The catheter of claim 5, wherein the first lumen and the second lumen each have one opening at the distal face of the elongated body and one side opening between the distal end of the elongated body and the balloon.

8. The catheter of claim 1, wherein the elongated body does not have any side openings between the balloon and the proximal end.

9. The catheter of claim 1, wherein the third lumen extends from the proximal end of the elongated body and ends in the opening in communication with the balloon.

10. The catheter of claim 1, further comprising a connecting element at the proximal end of the elongated body in which the first lumen, the second lumen, and the third lumen are fixed.

11. The catheter of claim 10, wherein the first lumen and the second lumen extend separately from each other as flexible tubes from a point proximal to the connecting element to the proximal end of the elongated body.

12. The catheter of claim 11, wherein the proximal ends of the first lumen and the second lumen are each connected to a syringe.

13. The catheter of claim 10, wherein the third lumen extends separately from the first and second lumens from a point proximal to the connecting element to the proximal end of the elongated body, wherein the proximal end of the third lumen has a closure device.
